(19) United States
(12) Patent Application Publication
(10) Pub. No.: US 2019/0160099 A1
(43) Pub. Date: May 30, 2019

(54) PHARMACEUTICAL COMPOSITION AND USE THEREOF

(71) Applicant: Henan Hualong Biotechnology Co., Ltd., Henan (CN)

(72) Inventors: Quanli Gao, Henan (CN); Dan Wei, Henan (CN)

(21) Appl. No.: 16/092,678

(22) PCT Filed: Mar. 15, 2017
(86) PCT No.: PCT/CN2017/076684

§ 371 Date: Oct. 10, 2018

(30) Foreign Application Priority Data
Jan. 23, 2017 (CN) 201710050114.0

(51) Int. Cl. A61K 35/17; A61K 39/395; C12N 5/0783; A61K 49/00; A61P 35/00

(52) U.S. Cl. CPC A61K 35/17; A61K 39/39558; A61K 2039/545; A61K 49/0008; A61P 35/00; C12N 5/0636

(57) ABSTRACT

Disclosed are a pharmaceutical composition comprising a PD-1 antibody and non-specifically amplified and activated T cells, and the use thereof. The pharmaceutical composition of the present invention comprises the PD-1 antibody and non-specifically amplified and activated T cells. The two components have a synergistic effect, can inhibit the growth of renal cancer cells and have a killing effect on renal cancer cells.

### TECHNICAL FIELD

The present invention relates to the field of anti-tumor drug research, in particular to a pharmaceutical composition and application thereof.

### BACKGROUND

Tumors are neoplasms formed in body under the effects of various tumorigenic factors as a result of abnormal proliferation and differentiation of cells in local tissue due to loss of normal regulation to their growth at the gene level. Once a neoplasm is formed, it will not cease to grow even when the pathogeny has been eliminated. Its growth is not regulated by normal physiological process in body, and instead it destroys normal tissues and organs, which is particularly obvious in malignant tumors. Compared with benign tumors, malignant tumors grow fast, exhibit infiltrating growth, are prone to bleeding, necrosis, ulcers, etc., often have distant metastases, result in body weight loss, weakness, anemia, loss of appetite, fever, and severe viscera function damaged, etc., and eventually cause the patient to die.

Renal cancer is a malignant disease that seriously harms human health. Most of the advanced renal cancers are insensitive to radiotherapy and chemotherapy and have a poor prognosis. Cellular immunotherapy has achieved good effects in biological treatment of tumors and has gradually become one of the important means for cancer treatment. In recent years, the immune function of cytokine-induced killer (CIK) cells has been widely studied. As a new type of broad-spectrum anti-tumor immune cells, CIK cells not only have powerful broad-spectrum anti-tumor effects, but also can regulate and enhance the immune function of patients suffering from tumors.

Programmed death-1 (PD-1), an important immunosuppressive molecule, is a member of CD28 superfamily, and is originally cloned from apoptotic mouse T cell hybridoma 2B4.11. PD-1 is mainly expressed in activated T cells and B cells, and has an inhibitory effect on cell activation, which is a normal homeostatic mechanism in immune system. Excessive activation of T/B cells can cause autoimmune disease, and therefore, the PD-1 acts as a guardrail in human body.

CN 105326893 A discloses an anti-cancer composition and formulation thereof. This composition comprises DC-CIK cells and Kangai injecta. By combining DC-CIK cells with Kangai injecta, in this invention can achieve a better effect even at a reduced dosage of the respective drugs, and can effectively treat cancers. The anti-cancer composition is not well-targeted and there is no strong evidence demonstrating that it has a significant therapeutic effect on cancer.

CN 105744955 A discloses a composition comprising an anti-CEACAM1 antibody, a composition comprising an antibody capable of inhibiting or blocking the interaction between PD-1 and its ligand, as well as the combined use of the compositions in the treatment of cancer. In this invention, the anti-PD-1 antibody is administrated alone or in combination with other antibodies. It is not well-targeted, and there is no strong evidence demonstrating that it has a significant therapeutic effect on cancer.

### SUMMARY OF THE INVENTION

In view of the current problems, the present invention provides a pharmaceutical composition and application thereof. The pharmaceutical composition comprises anti-PD-1 antibody and non-specifically amplified and activated T cells. The pharmaceutical composition has a significant therapeutic effect on renal cancer, achieving PR (partial remission) or nearly CR (complete remission) when evaluated according to the RESIST criteria, with an effective rate of 100% but very little side effects and without development of grade 3 or 4 adverse reactions.

To achieve this purpose, the present invention adopts the following technical solutions:

In one aspect, the present invention provides a pharmaceutical composition comprising anti-PD-1 antibody and non-specifically amplified and activated T cells.

In the present invention, the non-specifically amplified and activated T cells refer to a CD8CD4 mixed T lymphocyte population with broad-spectrum killing function obtained from the amplification and activation of CD8+ T cells and CD4+ T cells. Specifically, the non-specifically amplified and activated T cells are obtained by activation and amplification of T lymphocytes in the peripheral blood of patients by a variety of cytokines. They have positive immunomodulatory effects, and for example, they can secrete various cytokines such as IFN-γ, IL-2, etc., and can also inhibit immunosuppressive cells such as MDSC, etc. The non-specifically amplified and activated T cells can enter into tumor tissues when they are returned to patients. They exert an immunoregulatory effect in the microenvironment of tumor tissues. By inducing tumor cells to express PD-1 ligand, PD-L1, by the secreted cytokines such as IFN-γ and inhibiting the immunosuppressive cells in the tumor microenvironment, they exert an immunoregulatory effect. The anti-PD-1 antibody can reactivate the killing effect of T lymphocytes on tumor cells mainly by blocking the binding of PD-1 molecule on T lymphocytes in tumor tissues to PD-L1. However, due to the presence of immunosuppressive factors such as MDSCs and Treg cells in the tumor microenvironment, the reactivating effect of anti-PD-1 antibody on T lymphocytes is reduced. The non-specifically amplified and activated T cells can suppress the immunosuppressive cells in the tumor microenvironment, and thus enhance the effect of the anti-PD-1 antibody.

According to the present invention, the anti-PD-1 antibody is any anti-PD-1 antibody commercially available from commercial sources, and anti-PD-1 antibodies purchased from different commercial sources will not affect the efficacy of the pharmaceutical composition of the present application. In an embodiment, the anti-PD-1 antibody of the present invention can be purchased from Keytruda, and it is a prescription drug approved by the US FDA. The anti-PD-1 antibody has a concentration of 1-10 mg/kg, for example, 1 mg/kg, 1.5 mg/kg, 2 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 7.5 mg/kg, 8 mg/kg, 8.5 mg/kg, 9 mg/kg, 9.5 mg/kg or 10 mg/kg, preferably 1-5 mg/kg, further preferably 2 mg/kg, as well as specific values between the above values, and the specific values included in the ranges are no longer listed exhaustively in the present invention due to the limitation of space and for concise considerations.

According to the present invention, the non-specifically amplified and activated T cells have an amount of (3-12)×10⁹, for example, 3×10⁹, 3.1×10⁹, 3.2×10⁹, 3.3×10⁹, 3.5×10⁹, 3.8×10⁹, 4×10⁹, 4.2×10⁹, 4.5×10⁹, 4.8×10⁹, 5×10⁹, 5.3×10⁹, 5.5×10⁹, 5.8×10⁹, 5.83×10⁹, 6×10⁹, 6.5×10⁹, 7×10⁹, 7.5×10⁹, 8×10⁹, 8.5×10⁹, 9×10⁹, 9.5×10⁹, 10×10⁹, 10.5×10⁹, 11×10⁹, 11.5×10⁹ or 12×10⁹, preferably (6-10)×10⁹, further preferably 5.83×10⁹, as well as specific values between the above values, and the specific values included in the ranges are no longer listed exhaustively in the present invention due to the limitation of space and for concise considerations.

In a preferred embodiment, the non-specifically amplified and activated T cells are mixed T cells which are activated and amplified by CD3 in combination with recombinant human fibronectin (eg, RetroNectin), and comprise mainly CD8+ T cells and CD4+ T cells, and sometimes are referred to herein as CRAT cells (CD3-Retronectin activated T cells).

Preferably, the method for preparing the non-specifically amplified and activated T cells comprises the following steps:

(1) subjecting the patient's peripheral blood to a first centrifugation, storing the upper plasma layer obtained after the centrifugation in a refrigerator at 4° C., diluting the lower blood cells layer with normal saline, carefully layering the diluted blood cells over the lymphocyte separation medium, and subjecting to a second centrifugation;

(2) extracting mononuclear cells after the second centrifugation, washing them with normal saline, and counting the number of cells;

(3) inoculating the cells into a culture flask coated with recombinant human fibronectin (eg, RetroNectin), CD3 mAb and PBS after counting the number of cells, adding 50-60 mL of complete culture medium, placing them into a 5% CO2 incubator, and incubating the non-specifically amplified and activated T cells at 37° C.;

(4) the next day, observing under a microscope and supplementing with complete culture medium according to the state and quantity of cells;

(5) allowing the non-specifically amplified and activated T cells to mature over 10-20d, preferably 14d, washing them with normal saline, resuspending and then centrifuging at a low speed, discarding the supernatant, and resuspending the cells with normal saline containing 20% albumin.

In a specific embodiment of the present invention, RetroNectin is added to the culture system of lymphocytes to promote the cells to enter S phase from G1 phase, which would stimulate the cells to obtain tens of thousands of proliferation rates. When coating with Retronectin in combination with CD3 mAb, the Retronectin can not only involve in the adhesion, extension, differentiation and proliferation of cells, but also increase the contact and adhesion of CD3 mAb with the non-specifically amplified and activated T cells, i.e., increasing the effect of CD3 mAb, further enhancing the efficiency of amplification and cultivation of the non-specifically amplified and activated T cells.

Preferably, the first centrifugation in step (1) is performed at 500-1000 g, 4° C. for 10-20 min, preferably at 800 g, 4° C. for 15 min.

The centrifugation is performed at 500-1000 g, for example, 500 g, 550 g, 600 g, 650 g, 700 g, 750 g, 800 g, 850 g, 900 g, 950 g or 1000 g, as well as specific values between the above values, and the specific values included in the ranges are no longer listed exhaustively in the present invention due to the limitation of space and for concise considerations.

The centrifugation is performed for 10-20 min, for example, 10 min, 11 min, 12 min, 13 min, 14 min, 15 min, 16 min, 17 min, 18 min, 19 min, or 20 min, as well as specific values between the above values, and the specific values included in the ranges are no longer listed exhaustively in the present invention due to the limitation of space and for concise considerations.

Preferably, the volume ratio of the normal saline and the blood cells in step (1) is (1-3):1, for example, 1:1, 2:1 or 3:1, preferably 1:1, as well as specific values between the above values, and the specific values included in the ranges are no longer listed exhaustively in the present invention due to the limitation of space and for concise considerations.

Preferably, the second centrifugation in step (1) is performed at 500-1000 g, 16-23° C. for 10-20 min, preferably at 800 g, 20° C. for 17 min, as well as specific values between the above values, and the specific values included in the ranges are no longer listed exhaustively in the present invention due to the limitation of space and for concise considerations.

The centrifugation is performed at 500-1000 g, for example, 500 g, 550 g, 600 g, 650 g, 700 g, 750 g, 800 g, 850 g, 900 g, 950 g or 1000 g, as well as specific values between the above values, and the specific values included in the ranges are no longer listed exhaustively in the present invention due to the limitation of space and for concise considerations.

The centrifugation is performed at a temperature of 16-23° C., for example, 16° C., 17° C., 18° C., 19° C., 20° C., 21° C., 22° C., or 23° C., as well as specific values between the above values, and the specific values included in the ranges are no longer listed exhaustively in the present invention due to the limitation of space and for concise considerations.

The centrifugation is performed for 10-20 min, for example, 10 min, 11 min, 12 min, 13 min, 14 min, 15 min, 16 min, 17 min, 18 min, 19 min, or 20 min, as well as specific values between the above values, and the specific values included in the ranges are no longer listed exhaustively in the present invention due to the limitation of space and for concise considerations.

Preferably, the washing in step (2) is performed for 1-5 times, for example, 1 time, 2 times, 3 times, 4 times or 5 times, preferably 2-3 times, as well as specific values between the above values, and the specific values included in the ranges are no longer listed exhaustively in the present invention due to the limitation of space and for concise considerations.

Preferably, in step (3), the cells are counted and magnetically isolated of pure CD4+ T cells and CD8+ T cells, which are then mixed.

Preferably, the cell number ratio of the CD4+ T cells and CD8+ T cells is 1:1.

In the present invention, CD8+T, i.e. cytotoxic T cells (Tcs), can kill target cells through the release of granzymes and cytotoxic cytokines and through Fas pathway; CD4+T, i.e. helper T cells (Ths), can secrete a large variety of cytokines to maintain the immune response of Tcs. Coordination of these two effector cells is necessary for the effective maintenance of adaptive immunity. Therefore, the non-specifically amplified and activated T cells have stronger and longer-lasting killing efficiency in vivo than CD8+T or CIK alone.

Preferably, the complete culture medium in step (3) is serum-free medium supplemented with 800-1200 IU/mL of IL-2, 800-1200 IU/mL of IFN-γ, 100-200 IU/mL of gentamicin, 50-150 IU/mL of IL-1α and 1-8% of autologous plasma, preferably 1000 IU/mL of IL-2, 1000 IU/mL of IFN-γ, 160 IU/mL of gentamycin, 100 IU/mL of IL-1α and 5% of autologous plasma.

The IL-2 has a concentration of 800 IU/mL, 850 IU/mL, 900 IU/mL, 950 IU/mL, 1000 IU/mL, 1050 IU/mL, 1100 IU/mL, 1150 IU/mL, or 1200 IU/mL, as well as specific values between the above values, and the specific values included in the ranges are no longer listed exhaustively in the present invention due to the limitation of space and for concise considerations.

The IFN-γ has a concentration of 800 IU/mL, 850 IU/mL, 900 IU/mL, 950 IU/mL, 1000 IU/mL, 1050 IU/mL, 1100 IU/mL, 1150 IU/mL, or 1200 IU/mL, as well as specific values between the above values. The present invention would not exhaustively list the specific values included in the ranges due to the limitation of space and for the sake of conciseness.

The gentamycin has a concentration of 100 IU/mL, 120 IU/mL, 130 IU/mL, 140 IU/mL, 150 IU/mL, 160 IU/mL, 180 IU/mL, 190 IU/mL, or 200 IU/mL, as well as specific values between the above values, and the specific values included in the ranges are no longer listed exhaustively in the present invention due to the limitation of space and for concise considerations.

The IL-1α has a concentration of 50 IU/mL, 60 IU/mL, 70 IU/mL, 80 IU/mL, 90 IU/mL, 100 IU/mL, 110 IU/mL, 120 IU/mL, 130 IU/mL, 140 IU/mL, or 150 IU/mL, as well as specific values between the above values, and the specific values included in the ranges are no longer listed exhaustively in the present invention due to the limitation of space and for concise considerations.

The autologous plasma has a mass fraction of 1%, 2%, 3%, 4%, 5%, 6%, 7%, or 8%, as well as specific values between the above values, and the specific values included in the ranges are no longer listed exhaustively in the present invention due to the limitation of space and for concise considerations.

Preferably, in step (5), a centrifugation is performed after the washing, and the centrifugation is performed at 100-500 g, 4° C. for 5-16 min, preferably at 300 g, 4° C. for 8 min.

The centrifugation is performed at 100-500 g, for example, 100 g, 150 g, 200 g, 250 g, 300 g, 350 g, 400 g, 450 g, 500 g, 550 g, or 600 g, as well as specific values between the above values, and the specific values included in the ranges are no longer listed exhaustively in the present invention due to the limitation of space and for concise considerations.

The centrifugation is performed for 5-16 min, for example, 5 min, 6 min, 7 min, 8 min, 9 min, 10 min, 11 min, 12 min, 13 min, 14 min, 15 min, or 16 min, as well as specific values between the above values, and the specific values included in the ranges are no longer listed exhaustively in the present invention due to the limitation of space and for concise considerations.

Further preferably, the preparation method of the non-specifically amplified and activated T cells comprises the following steps:

(1) dispensing 50 mL of patient's peripheral blood (anticoagulated with heparin sodium) into two 50 mL centrifuge tubes, and subjecting to a centrifugation at 800 g, 4° C. for 15 min;

(2) after the centrifugation, drawing the upper plasma layer and placing into a 50 mL centrifuge tube and storing in a refrigerator at 4° C.;

(3) diluting the blood cells in the centrifuge tube with normal saline at a ratio of 1:1 and carefully layering the diluted blood cells over the lymphocyte separation medium, subjecting to a centrifugation at 800 G, 20° C. for 17 min.

(4) extracting mononuclear cells (i.e. the buffy coat) after the centrifugation, and placing them into a 50 mL centrifuge tube, washing with normal saline, subjecting to a centrifugation at 300 g, 4° C. for 8 min, washing 2 times;

(5) counting the number of cells, and magnetically isolating of pure CD4+ T cells and CD8+ T cells, and then mixing them with a cell number ratio of 1:1, inoculating the mixed cells into a culture flask coated with RetroNectin at 60 μL/flask, CD3 mAb at 15 μL/flask and PBS at 10 mL/flask, adding 50-60 mL of complete culture medium, which is serum-free medium supplemented with 1000 IU/mL of IL-2, 1000 IU/mL of IFN-γ, 160 IU/mL of gentamicin, 50-150 IU/mL of IL-1α and 5% of autologous plasma, placing into a 5% CO2 incubator, incubating the non-specifically amplified and activated T cells at 37° C.

(6) the next day, observing under a microscope and supplementing with appropriate amount of complete culture medium according to the state and quantity of cells;

(7) allowing the non-specifically amplified and activated T cells to mature over about 14d, performing quality control test in advance, collecting the cells if passing the test and in view of the doctor's prescription, washing them with normal saline for 3 times, resuspending and then centrifuging at a low speed, discarding the supernatant, and resuspending the cells with appropriate amount of normal saline containing 20% albumin.

In a second aspect, the present invention provides use of the pharmaceutical composition according to the first aspect in the preparation of an anti-tumor medication.

Preferably, the tumor is any one selected from the group consisting of lung cancer, breast cancer, renal cancer, or liver cancer, or a combination of at least two thereof, preferably renal cancer.

Preferably, the anti-tumor medication further comprises pharmaceutically acceptable adjuvants.

Preferably, the adjuvant is any one selected from the group consisting of excipient, diluent, carrier, flavoring agent, binder and filler, or a combination of at least two thereof.

In a third aspect, the present invention provides a method for detecting the inhibitory effect of the pharmaceutical composition according to the first aspect on renal cancer cells, comprising the following steps:

Transplanting the pharmaceutical composition according to the first aspect into a model organism suffering from renal cancer, and detecting tumor tissue masses of renal cancer in the model organism to judge the inhibitory effect of the pharmaceutical composition on renal cancer cells.

In the present invention, the model is used for detecting the inhibitory effect of the pharmaceutical composition on renal cancer cells, which is for non-therapeutic purpose. By transplanting the pharmaceutical composition into an immunodeficiency mouse suffering from renal cancer, which is a humanized mouse model that can effectively simulate pathological microenvironment in patients suffering from renal cancer, and performing several transplantations of pharmaceutical composition in this model to mimic a clinical treatment model and to insure a stable supply of the pharmaceutical composition, the therapeutic effect of the pharmaceutical composition on renal cancer is evaluated by tumor monitoring.

Preferably, the pharmaceutical composition is transplanted at a frequency of once every 2-5 weeks, for example, once every 2 weeks, once every 3 weeks, once every 4 weeks, or once every 5 weeks, preferably once every 3 weeks, as well as specific values between the above values, and the specific values included in the ranges are no longer listed exhaustively in the present invention due to the limitation of space and for concise considerations.

According to the present invention, the model organism is a conventional model organism in the art, and the model organism of the present invention may be any one selected from the group consisting of rabbit, mouse, cat, dog, or monkey, preferably an immunodeficiency mouse model.

Preferably, the detecting is performed by means of any one selected from the group consisting of measuring the size of the tumor tissue mass of renal cancer, weighing the tumor tissue mass of renal cancer, in vivo fluorescence imaging, or flow cytometry, and immunohistochemistry technology, or a combination of at least two thereof.

According to the present invention, said measuring the size of the tumor tissue mass of renal cancer, weighing the tumor tissue mass of renal cancer, in vivo fluorescence imaging, or flow cytometry, and immunohistochemistry technology are all conventional measurement method in the art. Those skilled in the art can make a selection according to actual needs, and no special limitation is made herein.

In a fourth aspect, the present invention provides use of the method according to the third aspect in evaluating inhibitory effect of the pharmaceutical composition on renal cancer cells.

In a fifth aspect, the present invention provides a method for treating a tumor in a subject, comprising: administering anti-PD-1 antibody to the subject and performing a treatment of non-specifically amplified and activated T cells.

In the above method, preferably, the anti-PD-1 antibody is administrated before, after or simultaneously with performing the treatment of non-specifically amplified and activated T cells.

Preferably, the anti-PD-1 antibody is administrated at a concentration of 1-10 mg/kg, preferably 1-5 mg/kg, and further preferably 2 mg/kg.

Preferably, the non-specifically amplified and activated T cells have an amount of (3-12)×10⁹, preferably (6-10)×10⁹, and further preferably 5.83×10⁹.

In a preferred embodiment, the treatment of non-specifically amplified and activated T cells is a process of activating and amplifying T cells by CD3 in combination with recombinant human fibronectin (eg, RetroNectin) to produce mixed T cells which comprise mainly CD8+ T cells and CD4+ T cells.

Preferably, the treatment of non-specifically amplified and activated T cells comprises the following steps:

(1) subjecting the patient's peripheral blood to a first centrifugation, storing the upper plasma layer obtained after the centrifugation in a refrigerator at 4° C., diluting the lower blood cells layer with normal saline, carefully layering the diluted blood cells over the lymphocyte separation medium, and subjecting to a second centrifugation;

(2) extracting mononuclear cells after the second centrifugation, washing them with saline, and counting the number of cells;

(3) inoculating the cells into a culture flask coated with recombinant human fibronectin, CD3 mAb and PBS after counting the numbers of the cells, adding 50-60 mL of complete culture medium, placing them into a 5% CO2 incubator, and incubating the non-specifically amplified and activated T cells at 37° C.;

(4) the next day, observing under a microscope and supplementing with complete culture medium according to the state and quantity of cells;

(5) allowing the non-specifically amplified and activated T cells to mature over 10-20d, preferably 14d, washing them with normal saline, resuspending and then centrifuging at a low speed, discarding the supernatant, and resuspending the cells with normal saline containing 20% albumin.

Preferably, the first centrifugation in step (1) is performed at 500-1000 g, 4° C. for 10-20 min, preferably at 800 g, 4° C. for 15 min;

Preferably, the volume ratio of the normal saline and the blood cells in step (1) is (1-3):1, preferably 1:1;

Preferably, the second centrifugation in step (1) is performed at 500-1000 g, 16-23° C. for 10-20 min, preferably at 800 g, at 20° C. for 17 min;

Preferably, the washing in step (2) is performed for 1-5 times, preferably 2-3 times;

Preferably, the complete culture medium in step (3) is serum-free medium supplemented with 800-1200 IU/mL of IL-2, 800-1200 IU/mL of IFN-γ, 100-200 IU/mL of gentamicin, 50-150 IU/mL of IL-1α and 1-8% of autologous plasma, preferably 1000 IU/mL of IL-2, 1000 IU/mL of IFN-γ, 160 IU/mL of gentamycin, 100 IU/mL of IL-1α and 5% of autologous plasma;

Preferably, in step (3), the cells are counted and magnetically isolated of pure CD4+ T cells and CD8+ T cells, which are then mixed;

Preferably, the cell number ratio of the CD4+ T cells and CD8+ T cells is 1:1;

Preferably, in step (5), a centrifugation is performed after the washing, and the centrifugation is performed at 100-500 g, 4° C. for 5-16 min, preferably at 300 g, 4° C. for 8 min.

Further preferably, the treatment of non-specifically amplified and activated T cells comprises the following steps:

(1) dispensing 50 mL of patient's peripheral blood (anticoagulated with heparin sodium) into two 50 mL centrifuge tubes, and subjecting to a centrifugation at 800 G, 4° C. for 15 min;

(2) after the centrifugation, drawing the upper plasma layer and placing the same into a 50 mL centrifuge tube and storing in a refrigerator at 4° C.;

(3) diluting the blood cells in the centrifuge tube with normal saline at a ratio of 1:1 and carefully layering the diluted blood cells over the lymphocyte separation medium, subjecting to a centrifugation at 800 G, 20° C. for 17 min.

(4) extracting mononuclear cells (i.e. the buffy coat) after the centrifugation, and placing them into a 50 mL centrifuge tube, washing with normal saline (centrifuging at 300 G, 4° C. for 8 min) for 2 times;

(5) counting the number of cells, magnetically isolating of pure CD4+ T cells and CD8+ T cells, and then mixing them with a cell number ratio of 1:1, inoculating the mixed cells into a culture flask coated with recombinant human fibronectin at 60 μL/flask, CD3 mAb at 15 μL/flask and PBS at 10 mL/flask, adding 50-60 mL of complete culture medium, which is serum-free medium supplemented with 1000 IU/mL of IL-2, 1000 IU/mL of IFN-γ, 160 IU/mL of gentamicin, 50-150 IU/mL of IL-1α and 5% of autologous plasma, placing into a 5% CO2 incubator, incubating the non-specifically amplified and activated T cells at 37° C.;

(6) the next day, observing under a microscope and supplementing with complete culture medium according to the state and quantity of cells;

(7) allowing the non-specifically amplified and activated T cells to mature over 14d, performing quality control test in advance, collecting the cells if passing the test and in view of the doctor's prescription, washing them with normal saline for 3 times (resuspending and then centrifuging at a low speed), discarding the supernatant, and resuspending the cells with normal saline containing 20% albumin.

In the above methods, preferably, the tumor is any one selected from the group consisting of lung cancer, breast cancer, renal cancer, or liver cancer, or a combination of at least two thereof, preferably renal cancer.

Preferably, the anti-PD-1 antibody and/or the non-specifically amplified and activated T cells are formulated with pharmaceutically acceptable adjuvants;

Preferably, the pharmaceutically acceptable adjuvant is any one selected from the group consisting of excipient, diluent, carrier, flavoring agent, binder and filler, or a combination of at least two thereof.

Compared with the prior art, the present invention has the following beneficial effects:

(1) The pharmaceutical composition of the present invention comprises anti-PD-1 antibody and non-specifically amplified and activated T cells, both of which can act synergistically to significantly inhibit the growth of renal cancer cells and have a killing effect on renal cancer cells.

(2) The pharmaceutical composition of the present invention has an obvious therapeutic effect on renal cancer, achieving PR (partial remission) or nearly CR (complete remission) when evaluated according to the RESIST criteria, with an effective rate of 100% but very little side effects and without development of grade 3 or 4 adverse reactions.

### DESCRIPTION OF THE DRAWINGS

FIG. 1 shows detection results of mature CRATs by using a flow cytometer, wherein FIG. 1(A) shows the results of CD3+CD4+ cells, FIG. 1(B) shows the results of CD3+CD8+ cells, PE and FITC are channels of the flow cytometer;

FIG. 2 shows the results of clinical trials after administration of the pharmaceutical composition of the present invention, wherein FIGS. 2(A)-(B) show tumor size of the metastases in brain and chest before treatment, FIGS. 2(C)-(D) show tumor size of the metastases in brain and chest after 2 months of treatment, and FIGS. 2(E)-(F) show tumor size of the metastases in brain and chest after 6 months of treatment;

FIG. 3 shows the results of clinical trials after administration of the pharmaceutical composition of the present invention, wherein FIGS. 3(A)-(E) show tumor size of the multiple metastases in bone, liver, brain, pancreas and right renal before treatment, FIGS. 3(F)-(J) show tumor size of the multiple metastases in each organ after 3 months of treatment, FIGS. 3(K)-(O) show tumor size of the multiple metastases after 5 months of treatment, and FIGS. 3(P)-(T) show tumor size of the multiple metastases after 11 months of treatment;

FIG. 4 shows the results of clinical trials after administration of the pharmaceutical composition of the present invention, wherein FIGS. 4(A)-(E) show tumor size of the multiple metastases in chest and abdomen before treatment, FIGS. 4(F)-(J) show tumor size of the multiple metastases in chest and abdomen after 45 days of treatment, FIGS. 4(K)-(O) show tumor size of the metastases after 3 months of treatment, and FIGS. 4(P)-(T) show tumor size of the metastases after 5 months of treatment;

FIG. 5 shows the results of clinical trials after administration of the pharmaceutical composition of the present invention, wherein FIG. 5(A), FIG. 5(C) and FIG. 5(E) show tumor size of the multiple metastases in lung before treatment, and FIG. 5(B), FIG. 5(D) and FIG. 5(F) show tumor size of the multiple metastases in lung after 2.5 months of treatment;

FIG. 6 shows the results of clinical trials after administration of the pharmaceutical composition of the present invention, wherein FIG. 6(A)-(D) show tumor size of the metastases in lung after 4 courses of the treatment.

### DETAILED DESCRIPTION

In order to further illustrate the technical measures adopted by the present invention and the effects thereof, the technical solutions of the present invention are further described below with reference to the accompanying drawings and specific embodiments, and however, the present invention is not limited to the scope of the embodiments.

In the examples, techniques or conditions, which are not specifically indicated, are performed according to techniques or conditions described in the literature of the art, or according to product instructions. The reagents or instruments for use, which are not indicated with manufacturers, are conventional products that are commercially available from formal sources.

### Example 1: Preparation of CRAT Cells

The preparation method of the CRAT cells comprises the following steps:

(1) 50 mL of patient's peripheral blood (anticoagulated with heparin sodium) was drawn and dispensed into two 50 mL centrifuge tubes, centrifuged at 800 g, 4° C. for 15 min;

(2) after the centrifugation, the upper plasma was drawn and placed into a 50 mL centrifuge tube and stored in a refrigerator at 4° C.;

(3) the blood cells in the centrifuge tube were diluted with normal saline at a ratio of 1:1 and the diluted blood cells were carefully layered over the lymphocyte separation medium, centrifuged at 800 g, 20° C. for 17 min.

(4) after the centrifugation, mononuclear cells (i.e. the buffy coat) were extracted and placed into a 50 mL centrifuge tube, washed with normal saline, centrifuged at 300 g, 4° C. for 8 min, and washed 2 times;

(5) the cells were counted, and magnetically isolated of pure CD4+ T cells and CD8+ T cells, which were then mixed with a cell number ratio of 1:1; then the mixed cells were inoculated into a coated culture flask (coated with RetroNectin at 60 μL/flask, CD3 mAb at 15 μL/flask and PBS at 10 mL/flask), 50-60 mL of complete culture medium was added, and the flask was placed into a 5% CO2 incubator at 37° C. for the culture of CRAT cells, wherein the complete culture medium was serum-free medium supplemented with 1000 IU/mL of IL-2, 1000 IU/mL of IFN-γ, 160 IU/mL of gentamicin, 50-150 IU/mL of IL-1α and 5% of autologous plasma;

(6) the next day, the flask was observed under a microscope and supplemented with appropriate amount of complete medium according to the state and quantity of cells;

(7) the CRAT cells became mature after 14d, a quality control test was performed in advance, the cells were collected if passing the test and in view of the doctor's prescription, washed with normal saline for 3 times (resuspended and then centrifuged at a low speed), the supernatant was discarded, and the cells were resuspended with appropriate amount of normal saline containing 20% albumin.

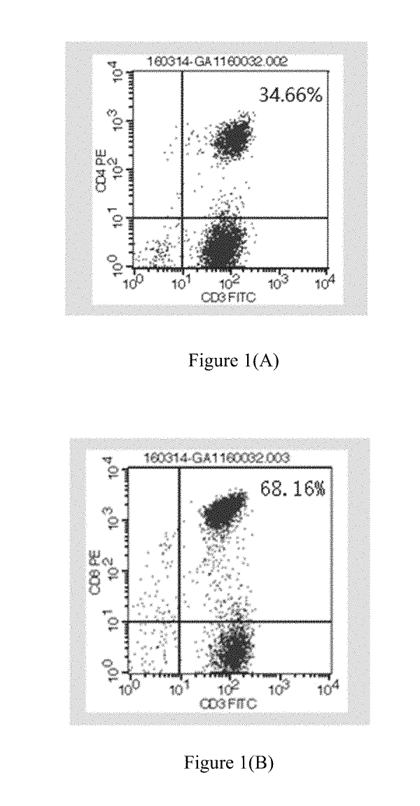

The cells were identified by flow cytometry. The results were shown in FIGS. 1(A)-1(B), which demonstrated that the content of CD3+CD4+ was 34.66%, and the content of CD3+CD8+ was 68.16%, which was in accordance with the distribution standard of cells.

### Example 2: Inhibitory Effect of the Pharmaceutical Composition on Renal Cells in Mouse Model

(1) A subcutaneous planting method was adopted to establish the mouse renal cancer model so as to facilitate observation of tumor growth. 25 normal 6-week-old nude mice were used, each mouse having a body weight of approximately 20 g. For each of the nude mouse, 0.2 ml of renal cancer cell suspension was subcutaneously injected into the right groin. After 10 d, when a 2-3 mm tumor was grown at the inoculation site, a mouse melanoma model was successfully created.

(2) Grouping and Treatment

After the modeling, the mice were randomly divided into 5 groups, including PBS control group (intraperitoneal injection with 1 mL sterile PBS), PD-1 mAb-treated group (intraperitoneal injection, 2 mg/kg), CRAT cells-treated group (5.83×10⁹ Bifidobacterium), and pharmaceutical composition-treated group, with 5 mice for each group. The next day after inoculation of tumor cells, dosing of the medication began and was conducted once every three weeks, for 4 consecutive cycles.

(3) Study on the Inhibitory Effect on Mouse Renal Cancer Cells

The next day after stopping dosing of the medication, the mice were weighed and sacrificed, and the tumor masses were dissected out and weighed. The tumor inhibition rate was calculated based on the average tumor weight. Tumor inhibition rate=[(tumor volume for the control group−tumor volume for the experimental group)/tumor volume for the control group]×100%.

(4) Experimental Results

The results showed that when comparing the treated groups with the PBS control group, the former significantly inhibited the growth of the mouse renal cancer cells, and the tumor inhibition rates of the PD-1 mAb-treated group, the CRAT cells-treated group and the pharmaceutical composition-treated group were 27%, 70%, and 100% respectively. Among them, there was significant difference (P<0.05) in the tumor inhibition rate when comparing the pharmaceutical composition-treated group with the other two groups, demonstrating that the pharmaceutical composition-treated group had higher inhibitory effect on renal cancer cells than that of the other groups.

### Example 3: Clinical Trial

Male, 50 years old. 2012.12 diagnosed with clear cell carcinoma of left renal with lung and bone metastases, underwent debulking surgery for the left renal cancer, followed by maintenance bisphosphonate. 2013.5 underwent γ-knife radiotherapy due to brain metastases. 2013.9-2014.10, treated with oral Sutent due to progression of lung metastases, during which and in 2013.11 underwent γ-knife radiotherapy and whole brain radiotherapy due to the occurrence of new metastases in brain. 2014.11-2015.5: switched to Everolimus due to progression of brain and lung metastases, during which and in 2014.4 underwent γ-knife stereotactic radiosurgery.

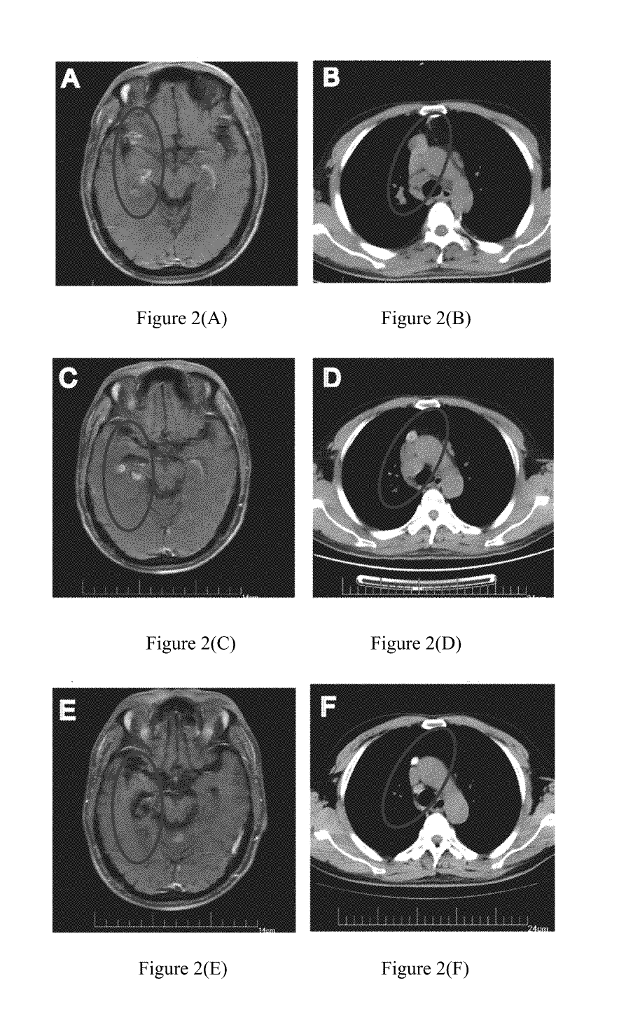

2015.6-2016.1: due to further progression of brain and breast metastases, anti-PD-1 antibody (Keytruda) was administrated at a dose of 2 mg/kg once every 3 weeks for a total of 8 times. CRAT cells were administrated at 5.83×10⁹ cells each time for a total of 11 times. The results were shown in FIG. 2. FIGS. 2(A)-(B) showed that the sum of the maximum diameters of metastases of the renal cancer was 6.7 cm before treatment. FIGS. 2(C)-(D) showed that brain and breast metastases were significantly reduced after 2 months of treatment. FIGS. 2(E)-(F) showed that the sum of the maximum diameters of brain and breast metastases after 6 months of treatment was 1.4 cm. The metastases were reduced by 79.1%. It was apparent that the brain and breast metastases were gradually reduced after 2 months of treatment. The efficacy lasted for 6.5 months and was evaluated as PR (partial remission).

2016.2-2016.6.8: brain metastases further progressed, supportive treatment was performed against symptoms, and the patient died on Jun. 8, 2016.

Adverse reactions: The highest body temperature after reinfusion of anti-PD-1 antibody was 37.8° C., and lasted for 36 hours (no antipyretic drugs were used). Mild fatigue (probably not related to the treatment).

### Example 4: Clinical Trial

Male, 66 years old. 2012.6 diagnosed with clear cell carcinoma of left renal with multiple bone metastases, underwent debulking surgery for the left renal cancer.

2012.7-2015.7: treated with oral Sorafenib. 2013.7 underwent radiotherapy (30 Gy/10f) to the third lumbar, 2015.4 underwent internal fixation with steel plate to the left humerus due to pathological fracture of the left humerus.

2015.7-2015.10: treated with oral Sunitinib due to progression of bone metastases. During this period, the tumors were further transferred to liver, brain, pancreas, and right adrenal glands. During the oral administration of Sunitinib, grade III thrombocytopenia and anemia and grade II scrotal skin ulcers were developed.

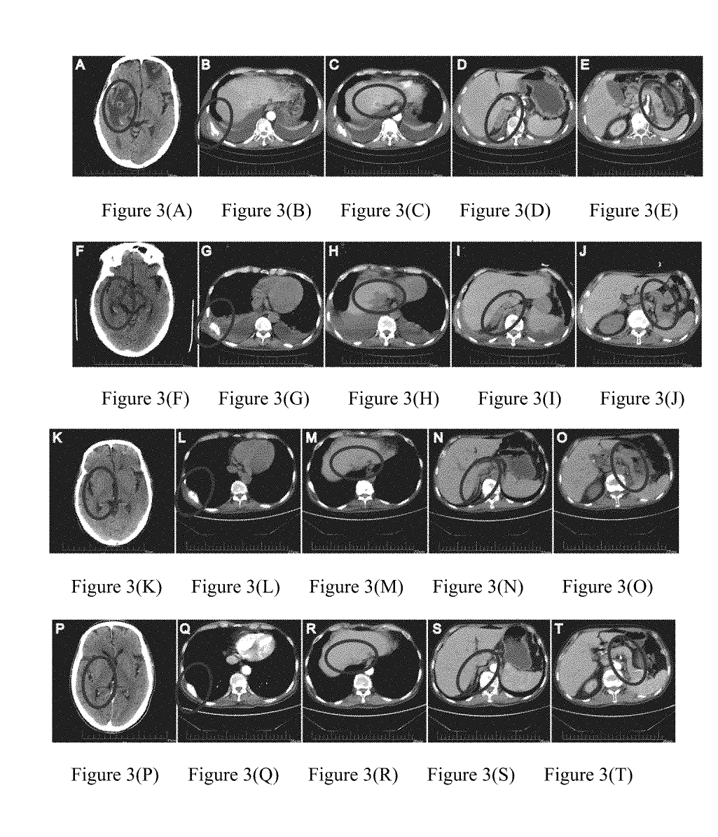

2015.7-2016.8: due to uncontrollable tumor progression, anti-PD-1 antibody (Keytruda) was administrated at a dose of 2 mg/kg once every 3 weeks for a total of 10 times, in combination with 5.83×10⁹ CRATs each time for a total of 10 times. The results were shown in FIG. 3. FIGS. 3(A)-(E) showed that the sum of the maximum diameters of metastatic tumors was 6.1 cm before treatment, and pleural effusion occurred. FIGS. 3(F)-(J) showed that the sum of the maximum diameters of metastatic tumors was significantly reduced after 3 months of treatment. FIGS. 3(K)-(O) showed that the sum of the maximum diameters of metastatic tumors was almost invisible after 5 months of treatment. FIGS. 3(P)-(T) showed that the metastatic tumors were disappeared and the pleural effusion was disappeared after 11 months of treatment. The efficacy lasted for more than 10 months and was evaluated as CR (complete remission).

Adverse reactions: The highest body temperature after the first two anti-PD-1 antibody reinfusions was 38.2° C., with grade 1 hypothyroidism.

### Example 5: Clinical Trial

Male, 79 years old. 2016.6 diagnosed with clear cell carcinoma of left renal, underwent radical resection for the left renal cancer.

2015.10-2016.3: treated with oral Axitinib due to breast and abdomen multiple metastases, poor efficacy, the tumor continued to progress, grade 3 adverse reactions of rash and diarrhea were developed.

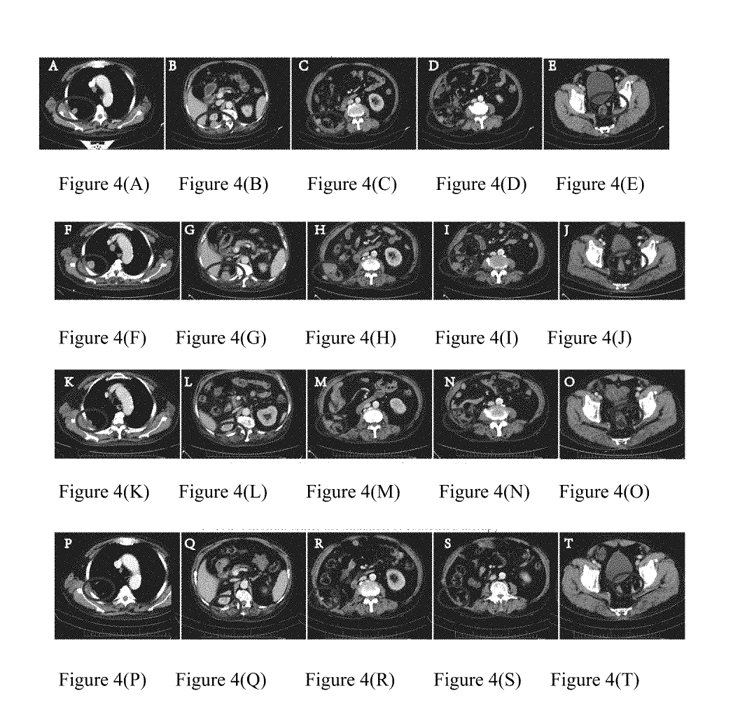

2015.6—till now: anti-PD-1 antibody was administrated at a dose of 2 mg/kg once every 3 weeks, in combination with 5.83×10⁹ CRAT cells each time, for a total of 6 times. Local radiotherapy was performed before immunotherapy in order to relieve the pain in third lumbar. The results were shown in FIG. 4. FIGS. 4(A)-(E) showed that the sum of the maximum diameters of metastatic tumors was 6.3 cm before treatment. FIGS. 4(F)-(J) showed that the sum of the maximum diameters of metastatic tumors was 0.6 cm after 45 days of treatment. FIGS. 4(K)-(O) showed that the sum of the maximum diameters of metastatic tumors was significantly reduced after 3 months of treatment. FIGS. 4(P)-(T) showed that the sum of the maximum diameters of metastatic tumors was 0.6 cm after 5 months of treatment. The tumors were reduced by 91.5%. The efficacy lasted for more than 7 months and was evaluated as PR (partial remission).

Adverse reactions: Several pieces of urticaria were developed on the skin after each time of anti-PD-1 antibody and reinfusion, and would disappear spontaneously without drug treatment.

### Example 6: Clinical Trial

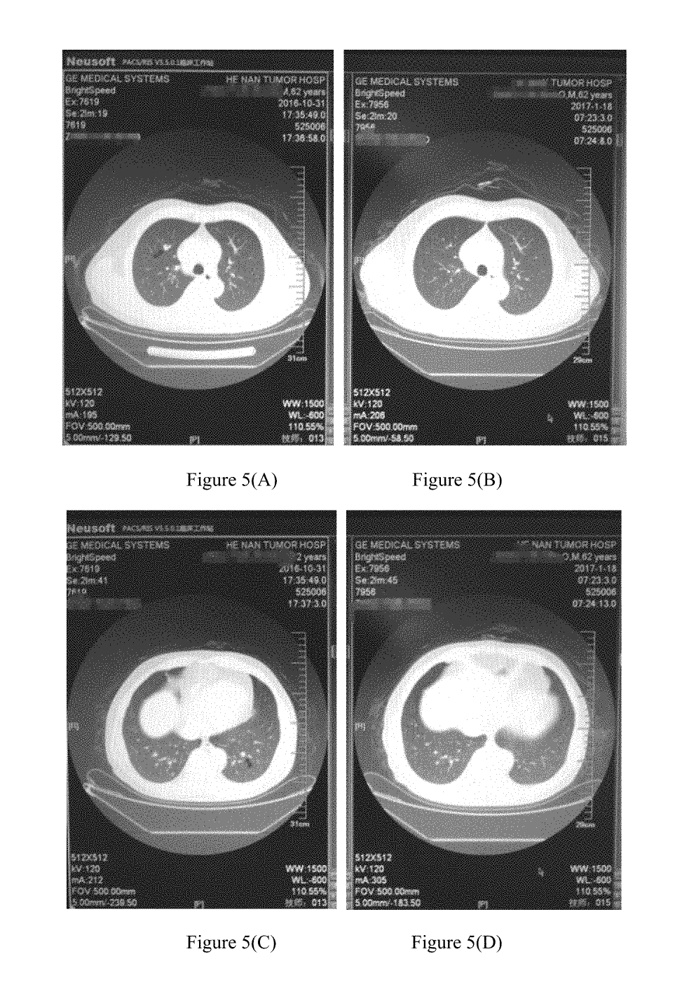

Male, 65 years old, right renal cancer stage 3, lung metastases developed 3 months after surgery, drug resistance and disease progression were developed after treatment with Sorafenib for 2 years and a half Anti-PD-1 antibody was administrated at a dose of 2 mg/kg once every 3 weeks, in combination with 5.83×10⁹ CRAT cells each time, for a total of 6 times. The results were shown in FIGS. 5 (A)-(F). The sum of the maximum diameters of tumors in lung metastases was 2.3 cm before treatment. The sum of the maximum diameters of tumors in lung metastases was reduced to 1.9 cm after 1 month of treatment. The tumors were reduced by 23.5%. The lung metastases began to shrink after 1 month of treatment, and the tumors were almost disappeared after 2 months of treatment. The efficacy was evaluated as CR (complete remission).

Adverse reactions: Side effects were low fever and fatigue after PD1 reinfusion, which were relieved after 3 days, and the patient said that the side effects were much smaller than those of Solafini.

### Example 7: Clinical Trial

Male, 58 years old. Underwent radical resection to the right renal cancer in November 2014, lung metastases were developed 11 months after the surgery.

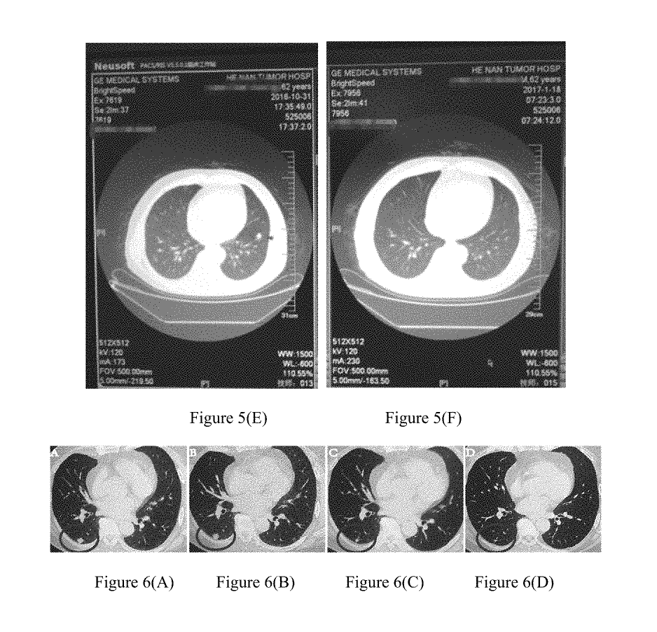

From November 2015 to July 2016: Anti-PD-1 antibody was administered at a dose of 2 mg/kg once every 3 weeks, in combination with 5.83×10⁹ of CRAT cells each time, for a total of 6 times. The results were shown in FIGS. 6(A)-(D). The sum of the maximum diameters of tumors in the lung metastases was 1.2 cm before treatment. The tumor cells in the lung metastases were substantially disappeared after 11 months of treatment. The efficacy lasted for more than 11 months and was evaluated as CR (complete remission).

Adverse reactions: No obvious adverse reactions.

In summary, the pharmaceutical composition of the present invention had a significant therapeutic effect on renal cancer through the synergistic action of PD-1 antibody and CRAT cells, achieving PR (partial remission) or nearly CR (complete remission) when evaluated according to the RESIST criteria, with an effective rate of 100% but very little side effects and without development of grade 3 or 4 adverse reactions.

The Applicant declares that detailed methods of the present invention have been described through the above examples, and however, the present invention is not limited to the above detailed methods. That is to say, it does not mean that the implementation of the present invention must rely on the above detailed methods. Those skilled in the art should understand that any improvement on the present invention, including the equivalent replacement of the raw materials or the addition of auxiliary components to the product of the present invention, and the selection of specific methods, etc., falls within the protection scope and the disclosure scope of the present invention.

1. A pharmaceutical composition comprising anti-PD-1 antibody and non-specifically amplified and activated T cells.

2. The pharmaceutical composition according to claim 1, wherein the anti-PD-1 antibody has a concentration of 1-10 mg/kg.

3. The pharmaceutical composition according to claim 1 or 2, wherein the non-specifically amplified and activated T cells have an amount of (3-12)×10⁹.

4. (canceled)

5. (canceled)

6. The pharmaceutical composition according to claim 1, wherein the non-specifically amplified and activated T cells are mixed T cells which are activated and amplified by CD3 in combination with recombinant human fibronectin, and comprise mainly CD8+ T cells and CD4+ T cells.

7. The pharmaceutical composition according to claim 1, wherein the method for preparing the non-specifically amplified and activated T cells comprises the following steps:
(1) dispensing 50 mL of patient's peripheral blood (anticoagulated with heparin sodium) into two 50 mL centrifuge tubes, and subjecting to a centrifugation at 800 G, 4° C. for 15 min;
(2) after the centrifugation, drawing the upper plasma layer and placing the same into a 50 mL centrifuge tube and storing in a refrigerator at 4° C.;
(3) diluting the blood cells in the centrifuge tube with normal saline at a ratio of 1:1 and carefully layering the diluted blood cells over the lymphocyte separation medium, subjecting to a centrifugation at 800 G, 20° C. for 17 min;
(4) extracting mononuclear cells (i.e. the buffy coat) after the centrifugation, and placing them into a 50 mL centrifuge tube, washing with normal saline (centrifuging at 300 G, 4° C. for 8 min) for 2 times;
(5) counting the number of cells, and magnetically isolating of pure CD4+ T cells and CD8+ T cells, and then mixing them with a cell number ratio of 1:1, inoculating the mixed cells into a culture flask coated with recombinant human fibronectin as 60 μL/flask, CD3 mAb at 15 μL/flask and PBS at 10 mL/flask, adding 50-60 mL of complete culture medium, which is serum-free medium supplemented with 1000 IU/mL of IL-2, 1000 IU/mL of IFN-γ, 160 IU/mL of gentamicin, 50-150 IU/mL of IL-1α and 5% of autologous plasma, placing into a 5% CO2 incubator, incubating the non-specifically amplified and activated T cells at 37° C.;
(6) the next day, observing under a microscope and supplementing with complete culture medium according to the state and quantity of cells;
(7) allowing the non-specifically amplified and activated T cells to mature over 14d, performing quality control test in advance, collecting the cells if passing the test and in view of the doctor's prescription, washing them with normal saline for 3 times (resuspending, then centrifuging at a low speed and discarding the supernatant), and resuspending the cells with normal saline containing 20% albumin.

8. (canceled)

9. (canceled)

10. A method for detecting the inhibitory effect of the pharmaceutical composition according to claim 1 on renal cancer cells, comprising the following steps:
transplanting the pharmaceutical composition according to claim 1 into a model organism suffering from renal cancer, and detecting tumor tissue masses of renal cancer in the model organism to judge the inhibitory effect of the pharmaceutical composition on renal cancer cells.

11. The method according to claim 10, wherein the pharmaceutical composition is transplanted at a frequency of once every 2-5 weeks;
the model organism is any one selected from the group consisting of rabbit, mouse, cat, dog, or monkey.

12. The method according to claim 10, wherein the detecting is performed by means of any one selected from the group consisting of measuring the size of the tumor tissue mass of renal cancer, weighing the tumor tissue mass of renal cancer, in vivo fluorescence imaging, or flow cytometry, and immunohistochemistry technology, or a combination of at least two thereof.

13. (canceled)

14. A method for treating a tumor in a subject, comprising: administering anti-PD-1 antibody to the subject and performing a treatment of non-specifically amplified and activated T cells.

15. The method according to claim 14, wherein the anti-PD-1 antibody is administrated before, after or simultaneously with performing the treatment of non-specifically amplified and activated T cells.

16. The method according to claim 14, wherein the anti-PD-1 antibody is administrated at a concentration of 1-10 mg/kg, preferably 1-5 mg/kg.

17. The method according to claim 14, wherein the non-specifically amplified and activated T cells have an amount of (3-12)×10⁹, preferably (6-10)×10⁹.

18. The method according to claim 14, wherein the treatment of non-specifically amplified and activated T cells is a process of activating and amplifying T cells by CD3 in combination with recombinant human fibronectin to produce mixed T cells which comprise mainly CD8+ T cells and CD4+ T cells.

19. The method according to claim 14, wherein the treatment of non-specifically amplified and activated T cells comprises the following steps:
(1) dispensing 50 mL of patient's peripheral blood (anticoagulated with heparin sodium) into two 50 mL centrifuge tubes, and subjecting to a centrifugation at 800 G, 4° C. for 15 min;
(2) after the centrifugation, drawing the upper plasma layer and placing the same into a 50 mL centrifuge tube and storing in a refrigerator at 4° C.;
(3) diluting the blood cells in the centrifuge tube with normal saline at a ratio of 1:1 and slowly adding the diluted blood cells to the upper layer of cell separation solution for lymphocytes, subjecting to a centrifugation at 800 G, 20° C. for 17 min;
(4) extracting mononuclear cells (i.e. the buffy coat) after the centrifugation, and placing them into a 50 mL centrifuge tube, washing with normal saline (centrifuging at 300 G, 4° C. for 8 min) for 2 times;
(5) counting the number of cells, and magnetically isolating of pure CD4+ T cells and CD8+ T cells, and then mixing them with a cell number ratio of 1:1, inoculating the mixed cells into a culture flask coated with recombinant human fibronectin at 60 μL/flask, CD3 mAb at 15 μL/flask and PBS at 10 mL/flask, adding 50-60 mL of complete culture medium, which is serum-free medium supplemented with 1000 IU/mL of IL-2, 1000 IU/mL of IFN-γ, 160 IU/mL of gentamicin, 50-150 IU/mL of IL-1α and 5% of autologous plasma, placing into a 5% CO2 incubator, incubating the non-specifically amplified and activated T cells at 37° C.;
(6) the next day, observing under a microscope and supplementing with complete culture medium according to the state and quantity of cells;
(7) allowing the non-specifically amplified and activated T cells to mature over 14d, performing quality control test in advance, collecting the cells if passing the test and in view of the doctor's prescription, washing them with normal saline for 3 times (resuspending and then centrifuging at a low speed), discarding the supernatant, and resuspending the cells with normal saline containing 20% albumin.

20. The method according to claim 14, wherein the tumor is any one selected from the group consisting of lung cancer, breast cancer, renal cancer or liver cancer, or a combination of at least two thereof.

21. The method according to claim 14, wherein the anti-PD-1 antibody and/or the non-specifically amplified and activated T cells are formulated with pharmaceutically acceptable adjuvants.

22. The pharmaceutical composition according to claim 1, wherein the method for preparing the non-specifically amplified and activated T cells comprises the following steps:
(1) subjecting the patient's peripheral blood to a first centrifugation, storing the upper plasma layer obtained after the centrifugation in a refrigerator at 4° C., diluting the lower blood cells layer with normal saline, carefully layering the diluted blood cells over the lymphocyte separation medium, and subjecting to a second centrifugation;
(2) extracting mononuclear cells after the second centrifugation, washing them with saline, and counting the number of cells;
(3) inoculating the cells into a culture flask coated with recombinant human fibronectin, CD3 mAb and PBS after counting the numbers of the cells, adding 50-60 mL of complete culture medium, placing them into a 5% CO2 incubator, and incubating the non-specifically amplified and activated T cells at 37° C.;
(4) the next day, observing under a microscope and supplementing with complete culture medium according to the state and quantity of cells;
(5) allowing the non-specifically amplified and activated T cells to mature over 10-20d, washing them with normal saline, resuspending and then centrifuging at a low speed, discarding the supernatant, and resuspending the cells with normal saline containing 20% albumin.

23. The pharmaceutical composition according to claim 22, wherein the first centrifugation in step (1) is performed at 500-1000 g, 4° C. for 10-20 min;
the volume ratio of the normal saline and the blood cells in step (1) is (1-3):1;
the second centrifugation in step (1) is performed at 500-1000 g, 16-23° C. for 10-20 min;
the washing in step (2) is performed for 1-5 times;
the complete culture medium in step (3) is serum-free medium supplemented with 800-1200 IU/mL of IL-2, 800-1200 IU/mL of IFN-γ, 100-200 IU/mL of gentamicin, 50-150 IU/mL of IL-1α and 1-8% of autologous plasma;
in step (3), the cells are counted and magnetically isolated of pure CD4+ T cells and CD8+ T cells, which are then mixed;
the cell number ratio of the CD4+ T cells and CD8+ T cells is 1:1;
in step (5), a centrifugation is performed after the washing, and the centrifugation is performed at 100-500 g, 4° C. for 5-16 min.

24. The method according to claim 14, wherein the treatment of non-specifically amplified and activated T cells comprises the following steps:
(1) subjecting the patient's peripheral blood to a first centrifugation, storing the upper plasma layer obtained after the centrifugation in a refrigerator at 4° C., diluting the lower blood cells layer with normal saline, adding the diluted blood cells to the upper layer of cell separation solution for lymphocytes, and subjecting to a second centrifugation;
(2) extracting mononuclear cells after the second centrifugation, washing them with saline, and counting the number of cells;
(3) inoculating the cells into a culture flask coated with recombinant human fibronectin, CD3 mAb and PBS after counting the numbers of the cells, adding 50-60 mL of complete culture medium, placing them into a 5% CO2 incubator, and incubating the non-specifically amplified and activated T cells at 37° C.;
(4) the next day, observing under a microscope and supplementing with complete culture medium according to the state and quantity of cells;
(5) allowing the non-specifically amplified and activated T cells to mature after 10-20d, washing them with normal saline, resuspending and then centrifuging at a low speed, discarding the supernatant, and resuspending the cells with normal saline containing 20% albumin.

25. The method according to claim 24, wherein the first centrifugation in step (1) is performed at 500-1000 g, 4° C. for 10-20 min;
the volume ratio of the normal saline and the blood cells in step (1) is (1-3):1;
the second centrifugation in step (1) is performed at 500-1000 g, 16-23° C. for 10-20 min;
the washing in step (2) is performed for 1-5 times;
the complete culture medium in step (3) is serum-free medium supplemented with 800-1200 IU/mL of IL-2, 800-1200 IU/mL of IFN-γ, 100-200 IU/mL of gentamicin, 50-150 IU/mL of IL-1α and 1-8% of autologous plasma;
in step (3), the cells are counted and magnetically isolated of pure CD4+ T cells and CD8+ T cells, which are then mixed;
the cell number ratio of the CD4+ T cells and CD8+ T cells is 1:1;
in step (5), a centrifugation is performed after the washing, and the centrifugation is performed at 100-500 g, 4° C. for 5-16 min.
